(19) United States
(12) Patent Application Publication
(10) Pub. No.: US 2012/0009660 A1
(43) Pub. Date: Jan. 12, 2012

(54) METHOD OF ASH REMOVAL FROM A BIOMASS

(75) Inventors: Raveendran Pottathil, La Jolla, CA (US); Joseph Stanton Bowers, JR., Palm Bay, FL (US); Gregory Dale Havemann, Pearland, TX (US)

(73) Assignee: PA, LLC, Melbourne, FL (US)

(21) Appl. No.: 13/157,293

(22) Filed: Jun. 9, 2011

Related U.S. Application Data
(60) Provisional application No. 61/353,121, filed on Jun. 9, 2010.

(51) Int. Cl. C07K 1/00; C10L 1/00; C07H 1/08; C07C 69/00; C08B 37/00; C07K 1/14

(52) U.S. Cl. 435/272; 435/274; 530/412; 536/128; 560/129; 44/307

(57) ABSTRACT

Disclosed herein is a process of reducing the ash content of a biomass feedstock or a biomass fraction. Embodiments include a process of reducing the ash content of a biomass feedstock or of a biomass fraction comprising: providing a biomass or biomass fraction; adding alcohol and acid to the biomass to yield a reaction mixture; separating the reaction mixture into a solid fraction and a liquid fraction by centrifugation; and distillating the liquid and collecting the solid to recover reaction reagents. The biomass can comprise aquatic species such as lemna.

### CROSS REFERENCE TO RELATED APPLICATION

This application claims priority under 35 U.S.C. 119(e) to U.S. Provisional Application No. 61/353,121, filed on Jun. 9, 2010, which is hereby incorporated by reference in its entirety.

### FIELD OF THE INVENTION

The present invention pertains to the field of processing biomass to produce fuels, chemicals and other useful products. Microcrop biomass is treated with alcohol and acid to reduce the ash content prior to further processing. Use of a prewash to remove minerals before acidifying the biomass is also provided as a method to improve acid pretreatment efficiency.

### BACKGROUND

The use of renewable biomass is becoming especially important as a feedstock for conversion to fuels, chemicals, electricity and heat. Most biomass consists of inorganic constituents (minerals), commonly referred to as ash.

Inorganic species naturally present in biomass play a dual role in terms of the thermochemical properties of biomass and its char. Inorganic species in biomass can significantly affect the temperature and extent of pyrolysis, and many are effective catalysts for combustion and gasification reactions of the resulting char.

Ash in biomass lowers the energy yield because the ash cannot be converted into usable products or energy. The existence of ash also complicates the conversion of biomass into liquid fuels by catalyzing the formation of gaseous products, decreasing the initial decomposition temperature, decreasing the pyrolysis rate, lowering the heating value of the liquids produced and increasing char formation.

Since the presence of inorganic species in biomass can alter downstream processing, a reduction of the ash content in the system is desirable before and during the processing steps. A reduction is necessary in both long-term protein products and biocrude products. In protein products, the desirable amount of ash content is less than 8% for animal feed such as tilapia, and less than 6% for human food additive products such as powdered milk, and can be required to be as low as 0.1% due to some governmental mandates. In biocrude products, the upper limit for ash content is 1.5% for combustion applications, and 2% for refinery, coking, and fermentation.

Treatment of microcrop biomass with alcohol and acid can be used to reduce the ash content prior to further processing. Use of a prewash to remove minerals before acidifying the biomass can additionally improve acid pretreatment efficiency.

### DESCRIPTION OF THE RELATED ART

Microcrop biomass represents an inexpensive and readily available feedstock for combustion and pyrolysis. The biomass can be fermented to produce alcohols and industrial chemicals, or chemically converted to other compounds, combusted to produce energy, co-fired with coal, or pyrolyzed to produce refined products. Microcrop biomass can also be a source of high value protein which is an essential additive in a number of animal feed formulations and human food products.

A variety of different solid and liquid biomass materials, and biomass materials as components of mixed wastes, are combusted in a wide range of combustion devices. The combustion process is used for heating and power generation purposes. Non-limiting examples of the general types of installations can be listed under the following broad categories, depending on the scale of operation:

a) Small stoves, fires and boilers employed for domestic cooking and heating.

b) Small and medium-scale boilers employed for commercial, process and district heat supply.

c) Small and medium scale boilers employed for combined heat and power, or power-only applications.

d) The co-firing of biomass materials in very large quantities with coal in large industrial and utility boilers.

Biomass materials can also contain noncombustible constituents, and the nature and behavior of these constituents significantly affect the design, operation and performance of the combustor and the boiler.

Most biomass materials have significant inorganic matter contents and many of the problems encountered with the combustion of biomass materials, or the co-combustion of biomass with coal, are associated with the nature and the behavior of the biomass ash components and the other inorganic constituents.

The key technical ash-related problems encountered by operators of biomass combustors and boilers have been associated with:

a) The formation of fused or partly-fused agglomerates and slag deposits at high temperatures within furnaces and stoves.

b) The formation of bonded ash deposits and accumulations of ash materials at lower temperatures on surfaces in the convective sections of boilers.

c) The accelerated metal wastage of furnace and boiler components due to gas-side corrosion under ash deposits, and due to ash particle impact erosion or ash abrasion of boiler components and other equipment.

d) The formation and emission of sub-micron aerosols and fumes.

e) Biomass ash impacts on the performance of flue gas cleaning equipment.

f) The handling, utilization, and disposal of ash residues from biomass combustion plants, and of the mixed ash residues from the co-firing of biomass in coal-fired boilers.

The inorganic materials in most solid fuels, including biomass, can generally be divided into two broad fractions: the inherent inorganic material and the extraneous inorganic material. The inherent inorganic material exists as part of the organic structure of the fuel, and is most commonly associated with the oxygen-, sulphur- and nitrogen-containing functional groups. These organic functional groups can provide suitable sites for the inorganic species to be associated chemically in the form of cations or chelates. Biomass materials are relatively rich in oxygen-containing functional groups, and a significant fraction of the inorganic material in some of the lower ash biomass fuels is commonly in this form. Inorganic species can also be present in very fine particulate form within the organic structure of some of the fuels, and behave essentially as an inherent component of the fuel. The extraneous inorganic material can be added to the fuel through geological processes, or during harvesting, handling and processing of the fuel. Biomass fuels, for instance, are commonly contaminated with soil and other materials, which have become mixed with the fuel during collection, handling and storage.

In acid treatments, nitric or hydrochloric acid can be used, but sulfuric acid is often favored because of its lower cost. However, pretreatment expenditures can still be large when sulfuric acid is used because substantial quantities of acid are required, and neutralization and disposal costs remain.

Methods to de-ash ban grass and wheat straw by chopping, crushing and extracting with water have previously described in Jensen P. A. et al., 2001. Biomass and Bioenergy 20: 431-446; Knudsen N. O. et al, 1998, Possibilities and evaluation of straw pretreatment, In Biomass for Energy and Industry, Proceedings of the International Conference, Wurzburg, Germany; and Turn et al, Removal of inorganic constituents of fresh herbaceous fuels: processes and costs, Proceedings Third Biomass Conference of the Americas, Montreal.

The use of dilute acids for de-ashing biomass has been described in Piyali Das et al, Influence of pretreatment for deashing of sugarcane bagasse on pyrolysis products, Biomass and Bioenergy, 27 (5) 2005 (445-457); Xinliang Liu et al, Removal of inorganic constituents from pine barks and switchgrass, Fuel Processing Technology, Article in Press 2011; and U.S. Pat. No. 7,208,160 B2.

All references are herein incorporated by reference.

### SUMMARY

Some embodiments include a process of reducing the ash content from a biomass feedstock or of a biomass fraction comprising: providing a biomass comprising an aquatic species; treating the biomass with an alcohol and acid; separating the reaction mixture into solid fraction and liquid fraction; and recovering reagents by collecting the solids and distillating the liquids. The biomass fraction can comprise a protein fraction, a carbohydrate fraction, a lipid-depleted carbohydrate fraction, a substantially lipid-free carbohydrate fraction or a lipid fraction derived from the biomass. The aquatic species can comprise lemna. The separating can comprise centrifugation of the mixture. The recovering can comprise phase separation and fractional distillation. The process can comprise mixing a material selected from the group consisting of a solid phase and a juice with an alcohol and an acid catalyst, to form a mixture, and separating the mixture into a liquid and a solid, whereby lipids and ash-forming components in the material are segregated into the liquid. The solid phase can comprise a first solid phase that can be generated from lysing the biomass and pressing the lysed biomass to yield a juice and first solid phase; a second solid phase that can be generated from filtering the juice to yield a filtered juice and a second solid phase; and a third solid phase that can be generated from clarifying the filtered juice to yield a clarified juice and third solid phase.

Some embodiments include a process of reducing the ash content of a biomass feedstock or of a biomass fraction. The biomass fraction can include but is not limited to a protein fraction, a carbohydrate fraction, a lipid-depleted carbohydrate fraction, a substantially lipid-free carbohydrate fraction or a lipid fraction derived from the biomass. The method can comprise the treatment of the biomass with an alcohol and acid, centrifugation of the reaction mixture to separate the solids from the liquid fraction, and collection of the solids and distillation of the liquids for recovery of reagents.

Some embodiments include a process of reducing the ash content of a wet biomass by removing components of the biomass that can give rise to ash prior to the drying procedure of the biomass. In particular exemplary embodiments, ethanol and hydrochloric acid can be added to the wet biomass. The material can be mixed at elevated temperature or pressure. In other embodiments, the mixing can be carried out at room temperature and atmospheric pressure. The mixture can enter a decanting apparatus, which spins the mixture at high speed, and the liquid therein can be forced through holes to separate the solid mass from the liquid. The solid mass retained within the decanting apparatus are substantially freed of lipids and will not give rise to ash. The separated liquid contains lipids and other substances that give rise to ash, and can be the subject of further processing to remove those substances following the drying procedure of the biomass.

Some embodiments include a process of reducing the ash content of the juice resulting from the pressing procedure of the raw biomass by removing components of the biomass that can give rise to ash. In particular exemplary embodiments, ethanol and hydrochloric acid can be added to the juice resulting from the pressing procedure of the raw biomass. The material can be mixed at elevated temperature or pressure. In other embodiments, the mixing is carried out at room temperature and atmospheric pressure. The mixture enters a decanting apparatus, which spins the mixture at high speed, and the liquid therein is forced through holes to separate the solid mass from the liquid. The solid mass retained within the decanting apparatus are substantially freed of lipids and will not give rise to ash. The separated liquid contains lipids and other substances that can give rise to ash, and can be the subject of further processing to remove those substances.

Some embodiments include a process of reducing the ash content by adding protease enzymes to any of the solid phases generated in the recovery process. The recovery process can comprise subjecting the unclarified juice to a filter press, adding water to the lysed biomass, sonicating the lysed biomass, and adding carbohydrate enzymes to the lysed biomass individually or in combination. The process can further include subjecting any of the solid phases to chromatography and solubilizing protein.

Some embodiments include a system for reducing the ash content comprising: a feedstock; an alcohol; a catalyst; a reaction vessel that can be adapted to facilitate a reaction among the feedstock, the alcohol, and a catalyst; and a vessel connected to the reaction chamber via a closable fluid connection. The catalyst can be acidic. The catalyst can comprise at least one catalyst selected from HBr, HCl, HCN, HF, and H₂S. The catalyst can be liquid. The alcohol can comprise at least one alcohol selected from methanol, ethanol, propanol, butanol, hexanol, heptanol, octanol, nonanol, and decanol. The alcohol can comprise methanol. The feedstock can comprise biocrude. The feedstock can comprise biomass. The biomass can comprise microcrop biomass or a biomass fraction. The biomass fraction can comprise a protein fraction, carbohydrate fraction, a lipid-depleted carbohydrate fraction, a substantially lipid-free carbohydrate fraction or a lipid fraction derived from the biomass. The reaction product can comprise a solid fraction and a liquid fraction. The reaction product can comprise unreacted alcohol. The vessel can comprise a separator adapted to separate the unreacted alcohol from the reaction product. The separator can be a distillation unit.

Some embodiments include a process of reducing the ash content from a biomass feedstock or of a biomass fraction comprising: providing a biomass comprising an aquatic species; lysing the biomass to generate a lysed biomass; treating the biomass with an alcohol and catalyst to generate a reaction mixture; separating the reaction mixture into a solid phase and a liquid phase; and recovering reagents by collecting the solids and distillating the liquids, wherein the ash content of the biomass is reduced by at least 40%. The providing step can further include producing the biomass fraction of an aquatic species on an industrial scale, and harvesting the biomass. The lysed biomass can include a protein fraction, a carbohydrate fraction, a lipid-depleted carbohydrate fraction, a substantially lipid-free carbohydrate fraction, or a lipid fraction derived from the biomass feedstock. The aquatic species can include at least one species of Lemna. The catalyst can include at least one acid. The catalyst can include at least one catalyst selected from HBr, HCl, HCN, HF, and H₂S. The acid can include an organic acid, including at least one of formic acid, acetic acid, oxalic acid, and glycolic acid. The acid can be nitric acid. The catalyst can be liquid. The alcohol can include at least one alcohol selected from methanol, ethanol, propanol, butanol, hexanol, heptanol, octanol, nonanol, and decanol. The alcohol can include methanol. The separating step can include pressing the lysed biomass to generate a juice. The separating step can include centrifugation of the mixture. The recovering can comprise phase separation and fractional distillation.

Some embodiments include mixing a material selected comprising at least one of the solid phase and the juice, with an alcohol and a catalyst to form a mixture, further separating the mixture into a liquid fraction and a solid fraction, whereby lipids and ash-forming components in the material are segregated into the liquid. The solid phase can include at least one of: a first solid phase generated from lysing the biomass and pressing the lysed biomass to yield a juice and first solid phase; a second solid phase generated from filtering the juice to yield a filtered juice and a second solid phase; and a third solid phase generated from clarifying the filtered juice to yield a clarified juice and third solid phase. The further separating step can include subjecting the mixture to centrifugation; collecting the solid fraction; and recovering reagents by distillating the liquid fraction. The centrifugation can be performed at about 3500 rpm, or at about 4000 rpm, or at about 4500 rpm. The centrifugation can be performed for a duration of about 15 minutes, or about 20 minutes, or about 30 minutes.

Some embodiments further include a removing step to reduce ash-forming components of the biomass prior to the separating step of the process, wherein the removing step includes: adding the mixture to a decanting apparatus; spinning the mixture at high speed in the decanting apparatus; generating a solid mass retained within the decanting apparatus that is substantially freed of lipids and ash-forming components; collecting a separated liquid that is forced through holes of the decanting apparatus and contains lipids and ash-forming components, and further processing the separated liquid to remove ash-forming components.

Some embodiments further include a removing step to reduce ash-forming components from the juice generated from the pressing step of the process, wherein the removing step includes: adding the mixture to a decanting apparatus; spinning the mixture at high speed in the decanting apparatus; generating a solid mass retained within the decanting apparatus that is substantially freed of lipids and ash-forming components; collecting a separated liquid that is forced through holes of the decanting apparatus and contains lipids and ash-forming components, and further processing the separated liquid to remove ash-forming components. The mixture can be generated at elevated temperature or pressure. The elevated temperature can be about 70° C., or about 80° C., or about 90° C. The elevated pressure can be about 10 psig, or about 15 psig. The mixture can be generated at room temperature and atmospheric pressure.

Some embodiments include adding a protease enzyme to the solid fraction or solids.

In some embodiments, the recovering step comprises: subjecting the liquid fraction to a filter press; adding water to the lysed biomass; sonicating the lysed biomass; and adding carbohydrate enzymes to the lysed biomass individually or in combination. The liquid phase or the solid phase can be further subjected to chromatography and solubilizing protein. In some embodiments, the treating step can include a transesterification process.

Some embodiments include a leaching step, comprising: suspending the biocrude in a solution selected from the group consisting of water and dilute acid for at least 20 hours to generate a mixture; subjecting the mixture to a filtering system; washing the mixture by adding water to the mixture and removing water or dilute acid; and drying the mixture to generate a biocrude with reduced ash content.

Some embodiments include a system of reducing the ash content from a biomass feedstock or of a biomass fraction of an aquatic species comprising: a reaction chamber suitable to facilitate a reaction among the biomass feedstock, an alcohol, and a catalyst; a vessel connected to the reaction chamber via a closable fluid connection; a lysing unit for lysing the biomass to generate a lysed biomass; a separating unit for separating the lysed biomass to generate a juice and a solid phase; a separator adapted to separate the unreacted alcohol from the reaction product; wherein processing the biomass in the system results in at least 40% reduction of the ash content of the biomass.

Some embodiments further include a first belt filter to facilitate separating a mother liquor and salts from the reaction among the biomass feedstock, the alcohol, and the catalyst; a second belt filter to facilitate addition of wash water and removal of water and dilute acid; a third belt filter to facilitate addition of wash water and removal of water and dilute acid; a fourth belt filter to facilitate addition of wash water and ammonia and removal of the filtrate; a fifth belt filter to facilitate addition of wash water and further removal of filtrate to generate a product; and a dryer to further process the product.

### BRIEF DESCRIPTION OF THE DRAWINGS

FIG. 1 shows a design drawing of an exemplary lipid co-counter current wash with neutralization process in accordance with an embodiment of the present invention.

FIG. 2 shows a flow diagram of an exemplary ash removal process for biomass in accordance with an embodiment of the present invention.

FIG. 3 shows an overview of a biomass growth and processing system in accordance with an embodiment of the present invention.

FIG. 4 shows an overview of a biomass treatment process involving lysing and/or pressing of a lemna biomass in accordance with an embodiment of the present invention.

### DETAILED DESCRIPTION

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Any discussion of the prior art throughout the specification should in no way be considered as an admission that such prior art is widely known or forms part of common general knowledge in the field.

Unless explicitly stated otherwise, throughout the description and the claims, the words “comprise(s),” “comprising,” “include(s),” “including,” and the like, are to be construed in an inclusive sense as opposed to an exclusive or exhaustive sense; that is to say, in the sense of “including, but not limited to.”

Microcrops are small aquatic plants and can include, but are not limited to, lemna. Lemna is a genus of free-floating aquatic plant from the duckweed family, also known as Lemnaceae family. These plants grow prolifically and have been used extensively as a model system for studies in plant biology, eco-toxicology, and in biopharmaceutical production. Species in this family are suitable as a source of animal feeds for agriculture and aquaculture.

Aquatic species, including microcrop species such as lemna, can be grown in a growth system that can comprise one or more bioreactors. The bioreactor(s) can be large-scale. The bioreactor(s) can be an open bioreactor, a closed bioreactor, or a semi-open bioreactor, or a combination thereof. The growth system can comprise a monitoring system. The bioreactor(s) can comprise a built-in monitoring system. The monitoring system can adjust the operation conditions including, but not limited to, the flow rate of nutrients and/or CO₂ into the bioreactor(s), light exposure, time and/or rate of harvest, or the like, or a combination thereof. Such adjustment can be made in real time or periodically. Such adjustment can optimize the aquatic species growth rates, yield, or both.

The growth rate, yield, or both can be further optimized by reducing the ash content of the biomass. Substantially ash-free biomass, also known as a substantially mineral-free biomass, is useful in downstream processes including but not limited to combustion, pyrolysis, and fermentation.

The process can include the use of a crude feedstock without complicated pre-treatment of, for example, extensive drying, degumming, or the like, or a combination thereof. As used herein, “crude” can indicate that the feedstock has not been subjected to complicated pre-treatment and/or it contains several ash-forming components. The process can include the use of a gaseous catalyst. The process can include recycling of catalysts and/or unreacted alcohol. The process can be suitable for large-scale production, or small-scale product.

As used herein, “industrial-scale” or “industrial scale” indicates that the method and system are commercially feasible or viable for processing a large amount of raw feedstock. Merely by way of example, the method and system described herein have the capacity to process at least 100 kg, or at least 500 kg, or at least 1000 kg, or at least 1500 kg, or at least 2000 kg, or at least 2500 kg, or at least 3000 kg or more of raw feedstock a day, and can run on a continuous mode or a batch mode.

The reaction agents can include at least one catalyst. The catalyst can increase the rate of reaction, and/or allow for more liberal feedstock standards, and/or limit the number of reaction steps, and/or enhance the yield of the reaction process, and/or increase the safety of production workers while reducing the environmental footprint of the production process.

The catalyst can be a basic catalyst. A basic catalyst can catalyze the reaction by removing a proton from the alcohol, and can make the alcohol more reactive. The catalyst can include, for example, NaOH, KOH, MeONa, NaOCH3, Na2CH2CH3, guanidines (e.g. TBD), metal complexes of the type M(3-hydroxy-2-methyl-4-pyrone)2(H2))2 where M=Sn, Zn, Pb, or Hg; liquid amine-based catalysts such as DEA, DMAE, TEMED, or TMAH, or the like, or a combination thereof.

The catalyst can be an acidic catalyst. An acid catalyst can catalyze the reaction by donating a proton to the alcohol, and can make the alcohol more reactive. The catalyst can include, for example, a Bronsted acid that can include a sulfonic or sulfuric type acid, H2S04, HCl, acetyl chloride, BF3, HBr, HCN, HF, H2S, or the like, or a combination thereof.

Embodiments of the present invention include a method for reducing the ash content of a biomass, comprising: placing the biomass in a vessel; adding an alcohol to the biomass in the vessel; adding a liquid acid; allowing the mixture to react for a period of time and at a temperature sufficient for the biomass reaction to occur; moving the mixture to a decantor, which can separate solids from liquids under centrifugation; and collecting the ash reduced biomass. Optionally, the liquid can be re-used in the reactor vessel for subsequent use; for example, two, three, four times, or more. Optionally, the liquid is transported to a distillation tower for recovery of the alcohol/acid reagent. The alcohol can comprise methanol or ethanol. The liquid acid can comprise HCl.

As used herein, “lysing” biomass encompasses mechanical or chemical procedures that disturb the organization of the organism on the level of individual cells or multicellular structures, so as to render the carbohydrates, proteins, and micronutrients present in the biomass organisms more available for downstream processing to purified protein, carbohydrate-containing materials, or micronutrient-containing fluids. Lysing can include, for example, chopping, shredding, smashing, pressing, tearing, lysis by osmotic pressure, or chemical treatments that degrade biological structures.

The concentration of the catalyst can be from about 0.01 M to about 100 M, or from about 0.1 M to about 50 M, or from about 0.5 M to about 20 M, or from about 0.8 M to about 10M, or from about 1 M to about 5 M, or from about 1 M to about 3 M. The concentration of the catalyst can be lower than about 100 M, or lower than about 50 M, or lower than about 30 M, or lower than about 20 M, or lower than about 10M, or lower than about 8 M, or lower than about 6 M, or lower than about 5 M, or lower than about 4 M, or lower than about 3 M, or lower than about 2 M, or lower than about 1 M. As used herein, “about” can indicate ±20% variation of the value it describes. The concentration of the catalyst can refer to the concentration of the effective catalyst composition(s). Merely by way of example, if a catalyst is generated in situ, the concentration of the catalyst can refer to that of the generated catalyst.

Merely by way of example, the catalyst can include gaseous HCl. HCl gas can be provided in the form of anhydrous methanolic HCl. HCl gas can be generated in situ by mixing other reaction agents, for example, the feedstock, with H2S04 and NaCl. The concentration of HCl gas can be from about 0.01 M to about 100 M, or from about 0.1 M to about 50 M, or from about 0.5 M to about 20 M, or from about 0.8 M to about 10 M, or from about 1 M to about 5 M, or from about 1 M to about 3 M. The concentration of the catalyst can be lower than about 100 M, or lower than about 50 M, or lower than about 30 M, or lower than about 20 M, or lower than about 10M, or lower than about 8 M, or lower than about 5 M. If HCl is generated in situ by, for example, H2S04 and NaCl, H2S04 and NaCl can be provided at a ratio of from about 100:1 to about 1:100, or from about 50:1 to about 1:50, or from about 20:1 to about 1:20, or from about 10:1 to about 1:10, or from about 5:1 to about 1:5. Merely by way of example, H2S04 can be provided at about 3 M, and NaCl at about 1 M, and HCl gas can be generated in situ by mixing the feedstock with such provided H2S04 and NaCl. As used herein, “in situ” means that a-gas-a catalyst is generated in the reaction chamber, and not added exogenously. Merely by way of example, HCl gas can be generated in situ by combining H2S04 and NaCl in the reaction chamber. It is understood that the example regarding HCl gas as the catalyst is provided for illustration purposes only, and is not intended to limit the scope of the application. Other catalysts, such as, basic catalysts, other acid catalysts, in form of a gas, liquid or solid, can be used in the process and/or the system described herein.

The reaction agents can include a feedstock. As used herein, the feedstock can refer to a mass source that can include at least one biomass. Merely by way of example, the mass source can include microalgae, yeast, bacteria, oil-seeds, plant matter, animal fats, or the like, or a combination thereof. The mass source can include aquatic species. The mass source may or may not be pre-treated before being used as the feedstock. The pre-treatment can include, for example, separation of the biomass from growth media, additional drying of the feedstock, physical or mechanical pulverization to increase the surface area of the feedstock, preheat, or the like, or a combination thereof.

The feedstock can comprise lower than about 90% (% w/w), or lower than about 80% (% w/w), or lower than about 70% (% w/w), or lower than about 60% (% w/w), or lower than about 50% (% w/w), or lower than about 40% (% w/w), or lower than about 30% (% w/w), or lower than about 20% (% w/w), or lower than about 10% (% w/w), or lower than about 8% (% w/w), or lower than about S % (% w/w), or lower than about 2% (% w/w), or lower than about 1% (% w/w), or lower than about 0.5% (% w/w) of water.

The reaction agents including the feedstock, the alcohol and the catalyst can be brought into contact in various manners. The reaction agents can be brought into contact simultaneously or at different times. Some of the reaction agents can be combined together before they are brought into contact with the rest of the reaction agents. Merely by way of example, the feedstock and the alcohol can be combined before they are brought into contact with the catalyst. As another example, the feedstock and the catalyst can be combined before brought into contact with the alcohol. As another example, the alcohol and the catalyst can be combined before they are brought into contact with the feedstock. If the feedstock includes multiple mass sources, the mass sources can be combined before or when the feedstock is brought into contact with other reaction agents including the alcohol the catalyst.

Any of the contact described above can include mixing. The treating step can include mixing. The mixing can be performed by a mixing apparatus including, for example, a mechanical mixer (e.g. a pedal), a vibrator, a circulating pump, a sonicator, or the like, or a combination thereof. The mixing can be performed by a combination of multiple number and/or types of mixing apparatuses. The mixing can be performed continuously (at frequency of infinity). Merely by way of example, if the mixing is performed by a pedal, the pedal can be rotating continuously; if the mixing is performed by a vibrator, the vibrator can be vibrating continuously; if the mixing is performed by a circulating pump, the circulating pump can be pumping continuously; if the mixing is performed by a sonicator, the sonicator can be running and generating sonication continuously. The mixing can be performed concomitantly. The mixing can be performed at a frequency from about 0.01 Hz to about 100 Hz, or from about 0.1 Hz to about 50 Hz, or from about 0.5 Hz to about 25 Hz, or from about 1 Hz to about 20 Hz. The mixing can be performed at a constant frequency. The mixing can be performed at variable frequencies. Merely by way of example, the mixing can be performed at frequencies varying accordingly to a sine function. The mixing can be performed by a combination of a multiple number and/or types of apparatuses, wherein each apparatus can run at the same frequency. The mixing can be performed by a combination of a multiple number and/or types of apparatuses, wherein at least one of the apparatuses can run at a different frequency than the other apparatuses.

The mixing can be performed at a strength. The strength can depend on and/or be controlled by the power of the mixing apparatus. The mixing can be performed a pre-selected mixing parameters including duration, strength, frequency, or the like, or a combination thereof. The pre-selected mixing parameters can be fixed, or variable, or a combination thereof. Merely by way of example, the duration and frequency of the mixing can include pre-selected fixed values, and the strength can vary as a pre-selected sine function. The mixing parameters can be adjusted in real time. Merely by way of example, the duration of the mixing can be adjusted in real time based on other real-time operation parameters, real-time measurements regarding, for example, quality and/or quantity of the reaction product, a user's instruction, an instruction from a centralized and/or remote control center, or the like, or a combination thereof.

Embodiments of the present invention include a method for clarifying the juice, which can include precipitation, centrifugation, and others, which are routinely known by those of skill in the art. The centrifugation parameters can be, for example, at least about 100 rpm, or at least about 250 rpm, or at least about 500 rpm, or at least about 750 rpm, or at least about 1000 rpm, or at least about 1500 rpm, or at least about 2000 rpm, or at least about 2500 rpm, or at least about 3000 rpm, or at least about 3500 rpm, or at least about 4000 rpm, or at least about 4500 rpm, or at least about 5000 rpm, or at least about 5000 rpm or higher. The residence time during the centrifugation process can be at least about 1 minute, or at least about 5 minutes, or at least about 10 minutes, or at least about 15 minutes, or at least about 20 minutes, or at least about 25 minutes, or at least about 30 minutes, or at least about 45 minutes, or at least about 60 minutes, or at least about 1.5 hours, or at least about 2 hours, or at least about 2.5 hours, or at least about 3 hours, at least about 3.5 hours, or at least about 4 hours, or at least about 5 hours, or at least about 6 hours, at least about 7 hours, or at least about 8 hours, or at least about 9 hours, or at least about 10 hours, or at least about 15 hours, or at least about 20 hours. Time ranges having as endpoints any of the times set forth above are specifically contemplated.

In certain embodiments of the present invention, the starting material is a biomass-derived fraction such as protein fraction, carbohydrate fraction, a lipid-depleted carbohydrate fraction, a substantially lipid-free carbohydrate fraction or a lipid fraction.

A “substantially mineral-free” biomass is a biomass that has been treated to remove at least a portion of the minerals present in the biomass prior to treatment. The initial biomass can comprise at least about 1% w/w, or at least about 2% w/w, or at least about 3% w/w, or at least about 4% w/w, or at least about 5% w/w, or at least about 10% w/w, or at least about 20% w/w, or at least about 25% w/w, or at least about 30% w/w, or at least about 40% w/w, or at least about 50% w/w, or at least about 60% w/w minerals. Substantially free is a biomass or fraction that has been treated wherein at least about 70% w/w, or at least about 75% w/w, or at least about 80% w/w, or at least about 85% w/w, or at least about 90% w/w, or at least about 95% w/w, or at least about 97% w/w, or at least about 98% w/w, or at least about 99% w/w of the minerals present in the untreated biomass are removed. The biomass is treated, for example, with an acid that will react with and solubilize the minerals. The solubilized minerals can be separated from the biomass to produce a substantially mineral-free biomass.

A biomass can be processed such that protein is extracted, leaving behind a carbohydrate-rich meal. This can yield a fraction comprising protein (“protein fraction”) and a fraction comprising carbohydrate (“carbohydrate fraction”). The carbohydrate fraction can be further processed to isolate lipids, thereby providing a fraction comprising lipid (“lipid fraction”) and a lipid-depleted carbohydrate fraction or a substantially lipid-free carbohydrate fraction.

Different components of an exemplary growth system including a bioreactor, a suitable aquatic species, a growth medium, a monitoring system and a harvest system are described in U.S. Provisional Application No. 61/171,036, filed Apr. 20, 2009, and PCT Application No. PCT/US10/31811, filed Apr. 20, 2010, all entitled “CULTIVATION, HARVESTING AND PROCESSING OF FLOATING AQUATIC SPECIES WITH HIGH GROWTH RATES;” and U.S. Provisional Application No. 61/314,736, filed Mar. 17, 2010, entitled “METHOD OF PROTEIN ISOLATION FROM LEMNAE.” All of the foregoing patent applications are incorporated herein by reference as though fully set forth in their entirety.

The reaction agents can include an alcohol. The alcohol can include, for example, methanol, ethanol, propanol, butanol, hexanol, heptanol, octanol, nonanol, decanol, or the like, or a combination thereof. The alcohol can include, for example, benzyl alcohol, iso-butyl alcohol, n-butyl alcohol, 2-ethyl hexanol, furfuryl alcohol, iso-propyl alcohol, n-propyl alcohol, or the like, or a combination thereof.

The biomass can be further processed based on considerations, such as, for example, suitability for further applications. Merely by way of example, the biomass can be processed using physical or chemical methods in downstream processing such as protein extraction, combustion, pyrolysis, and fermentation.

In certain embodiments, the reaction can be maintained at different incubation temperatures. The temperature of the incubation period can be maintained at about room temperature, or above room temperature. Merely by way of example, the temperature can be maintained above about 25° C., or above about 30° C., or above about 35° C., or above about 40° C., or above about 45° C., or above about 50° C., or above about 55° C., or above about 60° C., or above about 65° C., or above about 70° C., or above about 75° C., or above about 80° C.

In certain embodiments, the reaction can be maintained at and the mixture can be generated at elevated incubation temperatures. Merely by way of example, the elevated temperature can be above about 70° C., or above about 75° C., or above about 80° C., or above about 85° C., or above about 90° C., or above about 95° C., or above about 100° C., or above about 105° C., or above about 110° C., or above about 115° C., or above about 120° C., or above about 125° C.

In certain embodiments, the reaction can be maintained at and the mixture can be generated at elevated incubation temperatures.

Any of the steps in the process, including, but not limited to, providing the biomass, lysing, contacting or treating with reagents including alcohol and catalyst, separating, and recovering steps, can be performed at room temperature. Any of the steps in the process can be performed at a temperature other than the room temperature. Any of the steps in the process can be performed at about 0° C., or at about 10° C., or at about 20° C., or at about 30° C., or at about 40° C., or at about 45° C., or at about 50° C., or at about 55° C., or at about 60° C., or at about 65° C., or at about 70° C., or at about 75° C., or at about 80° C., or at about 85° C., or at about 90° C., or at about 95° C., or at about 100° C., or at about 110° C., or at about 120° C., or at a temperature higher than about 120° C. The contacting can be performed within a temperature range of about ±0° C., or at about ±2° C., or at about ±5° C., or at about ±10° C., or at about ±15° C., or at about ±20° C., or at about ±25° C., or at about ±30° C., or at about ±35° C., or at about ±40° C., or at about ±45° C., or at about ±50° C., or higher. Ranges of temperatures having as endpoints any of the above temperatures are specifically contemplated. Merely by way of example, the any of the steps in the process can be performed at temperatures from about 30° C. to about 90° C., or from about 40° C. to about 80° C., or from about 45° C. to about 75° C. Any of the steps in the process can be performed about a temperature about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100%, or about 110%, or about 120%, or higher than about 120% of the boiling point of the alcohol at a pressure. Ranges of temperatures having as endpoints any of the above temperatures are specifically contemplated. If the alcohol includes multiple alcohol compositions with different boiling points at the pressure, the boiling point of the alcohol can refer to the lowest one. Any of the steps in the process can be performed at a fixed temperature. Any of the steps in the process can be performed at a temperature varying during the steps. Any of the steps in the process can be performed at a pre-selected (e.g. fixed or variable) temperature, or at a temperature which can be adjusted in real time. Merely by way of example, any of the steps in the process can be performed at a temperature which can be adjusted in real time based on the real-time operation parameters, real-time measurements regarding, for example, quality and/or quantity of the reaction product, a user's instruction, an instruction from a centralized and/or remote control center, or the like, or a combination thereof. As used herein, the operation parameters can include, for example, temperature, and/or pressure, and/or duration of the contacting and any other features involved with the process (e.g. mixing, separating, distilling), or the like, or a combination thereof.

Any of the steps in the process, including, but not limited to, providing the biomass, lysing, contacting or treating with reagents including alcohol and catalyst, separating, and recovering steps, can be performed at about atmospheric pressure, or at a pressure higher than atmospheric pressure, or at a pressure about 100%, or about 110%, or about 120%, or about 150%, or about 200%, or about 250%, or about 300%, or about 400%, or about 500%, or higher than 500% of atmospheric pressure. Any of the aforementioned steps can be performed at a pressure lower than atmospheric pressure or at a pressure about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100% of atmospheric pressure. Pressure ranges of temperatures having as endpoints any of the above pressures are specifically contemplated. Any of the steps in the process can be performed at a fixed pressure. Any of the steps in the process can be performed at a pressure varying during the steps. Any of the steps in the process can be performed at a pre-selected (e.g. fixed or variable) pressure, or at a pressure which can be adjusted in real time. Merely by way of example, any of the steps in the process can be performed at a pressure which can be adjusted based on the real-time operation parameters, real-time measurements regarding, for example, quality and/or quantity of the reaction product, a user's instruction, an instruction from a centralize and/or remote control center, or the like, or a combination thereof.

The residence time during any part of the process can be optimized to increase the efficiency of the process. In certain exemplary embodiments, the residence time can be chosen to increase the recovery of soluble proteins in unclarified juice after passage through a filter press.

The residence time during any part of the process can be at least about 1 minute, or at least about 5 minutes, or at least about 10 minutes, or at least about 15 minutes, or at least about 20 minutes, or at least about 25 minutes, or at least about 30 minutes, or at least about 45 minutes, or at least about 60 minutes, or at least about 1.5 hours, or at least about 2 hours, or at least about 2.5 hours, or at least about 3 hours, at least about 3.5 hours, or at least about 4 hours, or at least about 5 hours, or at least about 6 hours, at least about 7 hours, or at least about 8 hours, or at least about 9 hours, or at least about 10 hours, or at least about 15 hours, or at least about 20 hours. Time ranges having as endpoints any of the times set forth above are specifically contemplated. The residence time during any part of the process can last a pre-selected period of time. The residence time during any part of the process can last a period of time which can be adjusted in real time. Merely by way of example, the residence time during any part of the process can last a period of time which can be adjusted in real time based on the real-time operation parameters, real-time measurements regarding, for example, quality and/or quantity of the reaction product, a user's instruction, an instruction from a centralized and/or remote control center, or the like, or a combination thereof.

Merely by way of example, the separation step can be performed by fraction distillation. The reaction product comprising the first component can be distilled in, e.g. a vaporizer, or the like. The distillation can be performed at a temperature from about 20° C. to about 200° C., or from about 100° C. to about 180° C., or from about 60° C. to about 100° C., or from about 70° C. to about 130° C., or from about 80° C. to about 120° C., or from about 90° C. to about 110° C. The distillation can be performed at a pressure below or above atmospheric pressure. The distillation can be performed at a pressure from about 0.01 bar to about 10 bar, or from about 0.1 bar to about 8 bar, or from about 0.3 bar to about 5 bar, or from about 0.5 bar to about 3 bar. The residue can be drained to a storage tank, and can be further processed.

In some embodiments, at least one of: the first solid phase, the second solid phase, the third solid phase, and the liquid phase, can be used to recover the biomass and/or biomass fraction which may include ash-forming components. The lysing can include using at least one of: a ball mill, a colloid mill, a knife mill, a hammer mill, a grinding mill, a puree machine, and a filter press. The pressing can include using at least one of a belt press, a fan press, a rotary press, a screw press, a filter press, and finisher press. Likewise, in some embodiments, the further pressing can be carried out using a screw press.

In some embodiments, the process further can include drying the biomass. The drying can be carried out using at least one of: a spin flash dryer, a spray dryer, a drum dryer, a flash dryer, a fluid bed dryer, a double drum dryer, and a rotary dryer. The decanting or filtering can be carried out using at least one of: a vibratory separator, a vibrating screen filter, a circular vibratory separator, a linear/inclined motion shaker, a decanter centrifuge, and a filter press. The vibratory separator can include at least one vibrating screen filter. The centrifuge can be a high-speed multi disc stack centrifuge,

Embodiments of the present application can include a system reducing the ash content from a biomass feedstock or of a biomass fraction of an aquatic species. The system can include a reaction chamber. The reaction chamber can include a reactor or a container, for example, a tube, cartridge, pipe, chamber, vat, tank, bag, bladder, balloon, liner, or the like. The reaction chamber can be in the shape essentially of a cylinder, a cube, a rectangular solid, a pyramid, a cone, a sphere, or the like, or a portion thereof, or a combination thereof. Merely by way of example, the reaction chamber can be in the shape essentially of a cylinder in the middle part and a half sphere at the top. As used herein, the shape does not indicate the orientation of the reaction chamber. Merely by way of example, if the reaction chamber is in the shape essentially of a cone whose cross-sectional area is tapering along its longitudinal axis, the portion with smaller cross-sectional area can be the top portion of the reaction chamber, or it can be the bottom portion of the reaction chamber. The reaction chamber can be of any suitable volume, for example, smaller than about 1 mL, from about 1 mL to about 100 mL, or from about 100 mL to about 250 mL, or from about 250 mL to about 500 mL, or from about 500 mL to about 1 L, or from about 1 L to about 10 L, or from about 10 L to about 100 L, or from about 100 L to about 250 L, or from about 250 L to about 500 L, or from about 500 L to about 1000 L, or from about 1000 L to about 5000 L, or from about 5000 L to about 10,000 L, or from about 10,000 L to about 50,000 L, or from about 50,000 L to about 100,000, or from about 100,000 L to about 250,000 L, or larger than about 250,000 L.

The reaction chamber can be made of a metal, glass, plastic, an alloy, or the like, or a combination thereof. The metal can include at least one material selected from stainless steel, aluminum, or the like, or a combination thereof. Merely by way of example, the reaction chamber can include a metal such as SS316 internally lined with glass, plastic, ceramic, fiber glass, Teflon, or other composites that are acid resistant. The reaction chamber can include a coating on at least part of its interior surface. As used herein, the interior surface of the reaction chamber can refer to its surface facing inside of the reaction chamber. The interior surface can be in direct contact with the reaction agents, or can be separated from the reaction agents by its coating. The coating can include a material selected from glass, plastic, ceramic, fiber glass, Teflon, or the like, or a combination thereof. The interior surface of the reaction chamber or its coating can have the properties of, for example, essentially non-reactivity with the reaction agents, corrosion resistance, heat insulation, or the like, or a combination thereof. The reaction chamber can include a coating on at least part of its exterior surface. As used herein, the exterior surface of the reaction chamber can refer to its surface facing outside of the reaction chamber. The exterior surface can be in direct contact with the ambient surrounding the reaction chamber, or can be separated from the ambient by its coating. The coating can include a material selected from glass, plastic, ceramic, fiber glass, Teflon, or the like, or a combination thereof. The exterior surface of the reaction chamber or its coating can have the properties of, for example, essentially nonreactivity with the ambient, corrosion resistance, heat insulation, or the like, or a combination thereof.

The reaction chamber can comprise an apparatus to achieve the desired temperature for the reaction to occur within the reaction chamber. The apparatus can include, for example, a jacket, a cavitation (or vacuum), a heater, or the like, or a combination thereof.

Embodiments of the present application are further illustrated by the following examples.

### EXAMPLES

The following non-limiting examples are provided to further illustrate embodiments of the present application. It will be appreciated by those of skill in the art that the techniques disclosed in the examples that follow represent approaches discovered by the inventors to function well in the practice of the application, and thus can be considered to constitute examples of modes for its practice. However, those of skill in the art will, in light of the present disclosure, appreciate that many changes can be made in the specific embodiments that are disclosed and still obtain a like or similar result without departing from the spirit and scope of the application.

### Example 1

### Ash Reduction of Biocrude Fraction

A process of reducing ash content from biomass is described in this example. The process was tested by experiments.

Biomass was processed to extract proteins and obtain a biocrude fraction. The biocrude fraction included biomass after protein extraction. The biocrude fraction contained mostly carbohydrates and some protein.

Table 1 shows the ash content of the starting biocrude (labeled “Starting Ash”) and the ash content of the biocrude after treatment (labeled “Final Ash Treated”). In this example, anhydrous HCl and hydrous HCl were tested; 65° C. and 25° C. were tested. Table 1 shows that the initial biocrude contained about 20% ash, and the final ash content was reduced to about 5%.

TABLE 1
Reductions in ash content

| | | | | | Starting | Final Ash |
|---|---|---|---|---|---|---|
| Sample | HCL | Methanol | Incubation | Duration | Ash | Treated |
| Biocrude | Anhydrous | Ratio | 65° C. | 60 minutes | 20.3% | 8.5% |
| | (no water) | 3 wt/1 wt | | | | |
| | HCL | | | | | |
| | 1N | | | | | |
| Biocrude | HCL | Ratio | 65° C. | 60 minutes | 20.3% | 8.3% |
| | (water) | 3 wt/1 wt | | | | |
| | 1N | | | | | |
| Biocrude | HCL | Ratio | 25° C. | 60+ minutes | 20.3% | 5.3% |
| | (water) | 3 wt/1 wt | | | | |
| | 1N | | | | | |
| Protein | Anhydrous | Ratio | 65° C. | 60 minutes | 12.4% | 3.92% |
| treated | 1N | 3 wt/1 wt | | | | |

### Example 2

### Ash Reduction of Protein Fraction

A process of reducing ash content from biomass fraction is described in this example. The process was tested by experiments. After whole lemna biomass was treated for extraction of proteins, the protein fraction was further processed. The last row in Table 1 shows the ash content of the starting protein fraction and the ash content of the protein fraction after treatment.

Table 1 shows that the initial biocrude contained about 20% ash, and the final ash content was reduced to about 5% to 8%.

### Example 3

### Biomass Growth and Processing System

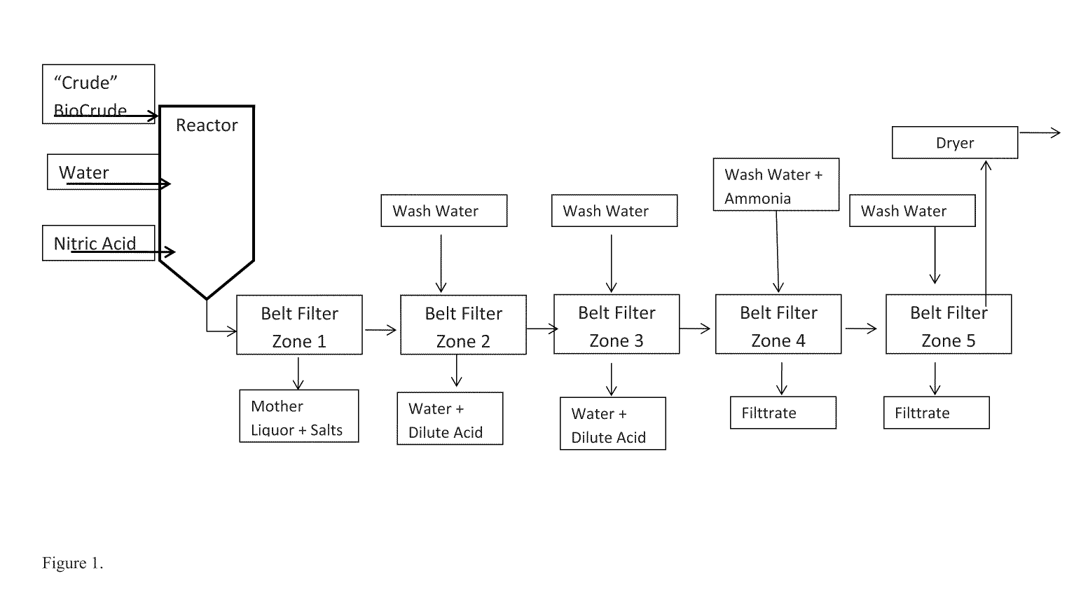

FIG. 1 shows an overview of the ash reduction system, which includes a co-counter current wash with neutralization process. Biocrude, water, and nitric acid are combined in a reactor. The reaction mixture enters the belt filter zone 1, from which the mother liquor and salts are removed. The remaining mixture is fed to the belt filter zone 2, and wash water is added to remove water and dilute acid. The washed mixture is fed to the belt filter zone 3, and wash water is added to remove water and dilute acid. The twice-washed mixture is fed to belt filter zone 4, and wash water and ammonia are added to remove the filtrate. The mixture is fed to belt filter zone 5, and wash water is added to removed more filtrate. The remaining mixture is fed to the dryer for further processing.

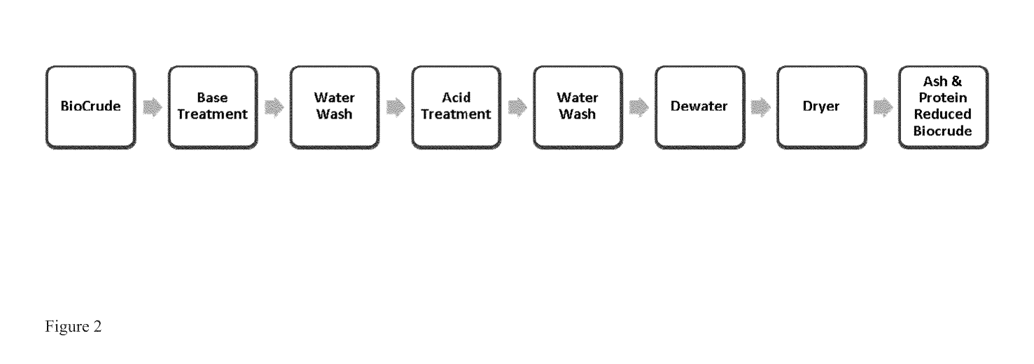

FIG. 2 shows an overview of the ash and protein reduction process from biocrude. The biocrude is treated with one or more bases. The mixture is washed with water, treated with one or more acids, and washed with water. The resulting mixture is subjected to a dewatering process, and then is fed to a dryer to generate a biocrude with a reduced ash and protein content.

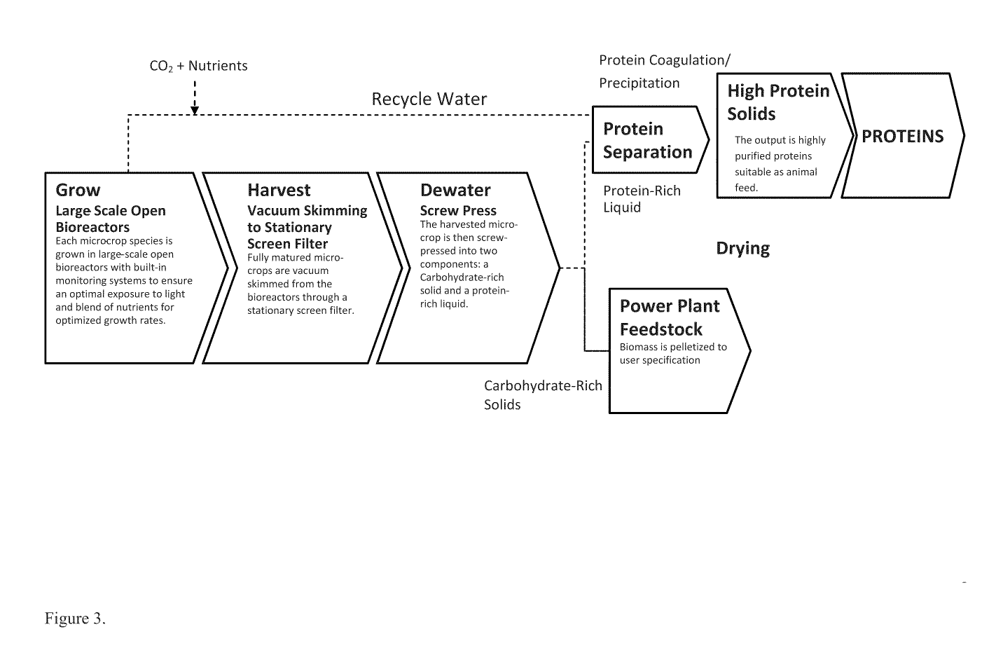

FIG. 3 shows an overview of the biomass growth and processing system. Microcrop species are grown in large-scale open bioreactors, in which a built-in monitoring system ensures an optimal exposure to light and blend of nutrients for optimized growth rates. Matured microcrops are vacuum skimmed from the bioreactors through a screen filter to be harvested, which are screw-pressed into two components: carbohydrate-rich solid and protein-rich liquid. After the dewatering step, the carbohydrate-rich solids are fed to the power plant as feedstock, and the biomass is pelletized to user specification. The protein-rich liquid is subjected to a protein coagulation and precipitation process, which results in the separation of high protein solids, highly purified and suitable as animal feed.

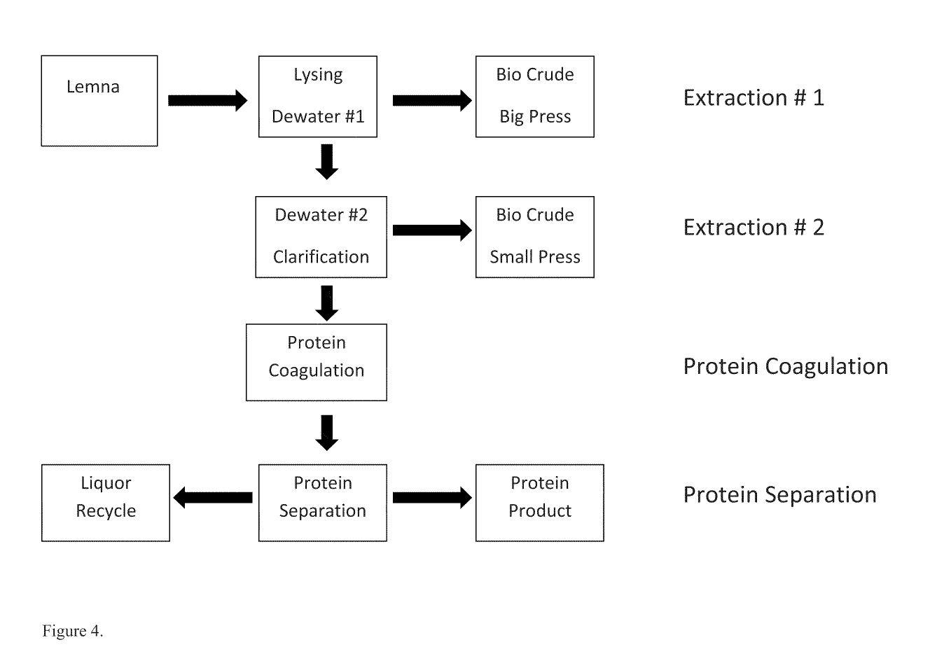

FIG. 4 shows an overview of the biomass treatment process comprising the lysing and/or pressing of a lemna biomass. The lemna biomass is also referred to as biomass slurry or raw feedstock. The lysing and/or pressing of a lemna biomass can generate a juice and a biocrude, which is referred to as “Bio Crude Big Press” in FIG. 4. The process is termed “Lysing Dewater #1” and “Extraction #1”. The juice can be filtered and/or clarified to generate another juice and further biocrude, which is referred to as “Bio Crude Small Press.” This process is termed “Dewater #2 Clarification” or “Extraction #2” in FIG. 4. After the dewatering, the solids can be combined to form a biocrude fraction; protein from the filtered and/or clarified juice can be coagulated to generate a protein-containing broth, which is termed “Protein Coagulation” in FIG. 4. The broth can be separated to generate a protein product and a liquor, which is termed “Protein Separation” in FIG. 4.

The ash removal treatment can be conducted on the biocrude fraction prior to drying.

The ash removal treatment can be conducted on the protein fraction prior to drying.

### Example 4

### Low pH Washings

Bio-crude was washed with low pH (2.0) water (sulfuric acid). Table 2 summarizes the effect of washing on ash level in processed biocrude.

TABLE 2
Reductions in ash content

| | Ash | Protein | Fat | Fiber | Total Carb |
|---|---|---|---|---|---|
| Sample | (%) | (%) | (%) | (%) | by Calc (%) |
| Biocrude | 9.2 | 24.3 | 4.5 | 18.8 | 61.9 |
| First wash | 3.4 | 23.9 | 4.1 | 22.5 | 68.6 |
| Second | 2.5 | 23.9 | 4.9 | 23.7 | 68.7 |
| wash | | | | | |

The ash level decreased with each additional wash step. The protein and fat levels were not significantly affected, while the relative fiber and carbohydrate content increased.

### Example 4

### Transesterification Process

The transesterification process was effective when applied to algae in previous studies. The use of the simplified process is further described in WO 2010/077685, which describes a transesterification process whereby triglycerides can be converted into methyl ester biodiesel, and for which a gaseous catalyst can be used as part of the esterification/transesterification process.

The effect of the transesterification process for ash removal in lemna was examined. Methanol was added to the feedstock, in the presence of HCl or HCl and reverse osmosis water.

The results are summarized in Table 3. “TE” refers to transesterification, and “RO” refers to reverse osmosis.

TABLE 3
Application of the simplified process to de-ashing biocrude

| | Protein | Ash | Fat | Fiber | Yield | Total Carb | |
|---|---|---|---|---|---|---|---|
| Sample | (%) | (%) | (%) | (%) | (%) | by Calc (%) | Treatment |
| Control | 22.0 | 8.0 | 3.8 | 20.3 | NA | 66.2 | NA |
| MeOH/HCl | 25.9 | 4.0 | 1.0 | 31.0 | 73.5 | 69.1 | TE Process |
| 50/50 | 26.7 | 4.2 | 4.8 | 32.0 | 68.5 | 64.3 | TE Process/ |
| MeOH—HCl/RO | | | | | | | H₂O |
| RO/HCl | 26.2 | 4.8 | 3.2 | 26.3 | 73.0 | 65.7 | Aq HCl |

When applied to lemna, ash levels decreased by about 50% and overall yields also decreased. The use of aqueous HCl produced similar results in that ash levels decreased by about 50% and overall yields were lower compared to P1.

### Example 5

### Acid Soaking

Leaching, or soaking, involves suspending the biocrude in water or dilute acid for a period of time, usually 20 hours or longer, followed by filtration, washing, and drying steps.

Sulfuric, phosphoric and hydrochloric acids were evaluated using the following steps. The biomass was suspended in a volume of water (control) or dilute acid. The mixture was then incubated at room temperature for approximately one day. The solids were collected by filtration using a stainless steel mesh, and washed by rinsing with two aliquots of water that were several times the volume of the starting material mass.

Results are summarized in Table 4.

TABLE 4
Removal of ash using water, H₂SO₄ and H₃PO₄ soaking

| | Ash | Protein | Fat | Fiber | Total Carb | Yield |
|---|---|---|---|---|---|---|
| Sample | (%) | (%) | (%) | (%) | by Calc (%) | (%) |
| Biocrude | 7.9 | 19.5 | 3.7 | 21.2 | 68.9 | NA |
| H₂O | 5.8 | 17.3 | 4.7 | 23.8 | 72.2 | 94.5 |
| 0.2N H₂SO₄ | 2.2 | 18.3 | 4.8 | 29.3 | 74.4 | 79.5 |
| 0.4N H₂SO₄ | 1.9 | 18.5 | 4.3 | 26.2 | 75.3 | 63.6 |
| 0.8N H₂SO₄ | 1.2 | 20.8 | 3.3 | 39.3 | 74.7 | 64.3 |
| Biocrude | 9.3 | 17.4 | 2.8 | 22.7 | 70.5 | NA |
| H₂O | 6.5 | 15.2 | 4.5 | 24.8 | 73.8 | 96.3 |
| 0.2N H₃SO₄ | 4.8 | 14.3 | 3.6 | 25.7 | 77.3 | 95.6 |
| 0.4N H₃SO₄ | 6.1 | 14.5 | 4.7 | 35.2 | 74.8 | 88.8 |
| 0.8N H₃SO₄ | 11.2 | 14.2 | 6.0 | 36.5 | 68.6 | 75.2 |

Leaching with H₂O, H₂SO₄ and H₃PO₄ resulted in lower ash levels. Leaching with high concentrations of H₂SO₄ and H₃PO₄ resulted in lower yield levels.

### Example 6

### Organic Acids

Several organic acids were evaluated as de-ashing acids. Formic, acetic, oxalic and glycolic acids were evaluated. The organic acids were examined in the following combinations: alcohol solvent with an alcohol wash, alcohol solvent with a water wash, and water solvent with a water wash.

TABLE 5
Organic Acids

| | Ash | Protein | Fat | Yield |
|---|---|---|---|---|
| Sample | (%) | (%) | (%) | (%) |
| Ethanol-Ethanol Wash | | | | |
| Control | 7.8 | 18.0 | 5.6 | NA |
| HOAc, 0.4N | 8.2 | 19.2 | 2.1 | 92.0 |
| Formic, 0.4N | 8.5 | 19.2 | 1.8 | 92.0 |
| Oxalic, 0.4N | 8.5 | 18.9 | 1.4 | 100 |
| Glycolic, 0.4N | 8.9 | 19.4 | 1.8 | 100 |
| Ethanol-Water Wash | | | | |
| HOAc, 0.4N | 6.3 | 17.6 | 2.1 | 86.0 |
| Formic, 0.4N | 6.0 | 17.7 | 1.8 | 86.0 |
| Oxalic, 0.4N | 5.6 | 17.8 | 1.7 | 80.0 |
| Glycolic, 0.4N | 6.3 | 17.8 | 1.7 | 80.0 |
| Water-Water Wash | | | | |
| HOAc, 0.4N | 4.9 | 16.8 | 2.9 | 86.0 |
| Formic, 0.4N | 4.1 | 16.5 | 4.0 | 87.0 |
| Oxalic, 0.4N | 2.3 | 17.9 | 2.8 | 79.0 |
| Glycolic, 0.4N | 2.5 | 17.0 | 2.8 | 89.0 |

The organic acids, such as oxalic and glycolic acid, were effective in de-ashing the biocrude without significantly lowering the yield.

### Example 7

### Nitric Acid

The data in Table 6 were compiled from runs using 3 different batches of biocrude and 5 different concentrations of nitric acid. The batches were heated to 60° C. and stirred for 30 minutes.

TABLE 6
De-ashing results with nitric acid on biocrude

| Acid | Average Ash | Average |
|---|---|---|
| Concentration | Level (%) | Yield (%) |
| 0.35N | 1.35 | 65 |
| 0.25N | 1.95 | 68 |
| 0.10N | 3.68 | 79 |
| 0.05N | 4.98 | 85 |
| 0.00N | 6.44 | 88 |

The results indicated that nitric acid is an effective de-ashing acid. Nitric acid residues produced nitrogen oxides when burned, and left no ash residue and produced no sulfur or chlorides.

### Example 8

### General De-Ashing Procedure

To perform the general de-ashing procedure, the following were combined: dry biocrude, reverse osmosis water, and an appropriate amount of 70% nitric acid determined by the acid concentration shown in Table 6. The slurry was stirred while being heated to 60° C. and was held at 60° C. for 30 minutes. The hot slurry was vacuum-filtered through a stainless steel screen, and washed with two separate portions of reverse osmosis water. The filter cake was pulled down well to remove as much water as possible, and dried in an oven overnight.

The results show that lemna can be effectively de-ashed using dilute aqueous solutions of most mineral acids and moderately de-ashed with aqueous solutions of organic acids, such as oxalic and glycolic acids at moderate temperatures (40-80° C.) and under one hour stir time. Nitric acid does not leave halogen or sulfur residues in the de-ashed product, which can be important in certain usage of de-ashed lemna, such as, for example, as a fuel or as a feedstock for other thermal transformations including pyrolysis or coking. Good de-ashing conditions can also be good conditions for hydrolyzing hemi-cellulose as well as other sugars and some proteins.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about” or “substantially”. For example, “about” or “substantially” can indicate ±1%, ±2%, ±3%, ±4%, ±5%, ±6%, ±7%, ±8%, ±9%, ±10%, ±11, %, ±12%, ±13%, ±14%, ±15%, or ±20% variation of the value it describes, unless otherwise stated. Accordingly, in some embodiments, the numerical parameters set forth in the written description and attached claims are approximations that can vary depending upon the desired properties sought to be obtained by a particular embodiment. In some embodiments, the numerical parameters are construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Each of the patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

We claim:

1. A process of reducing the ash content from a biomass feedstock or a biomass fraction of an aquatic species comprising:
providing the biomass;
lysing the biomass to generate a lysed biomass;
treating the biomass with an alcohol and a catalyst to generate a reaction mixture;
separating the reaction mixture into a solid phase and a liquid phase; and
recovering reagents by collecting the solids and distillating the liquids,
wherein the ash content of the biomass is reduced by at least 40%.

2. The process of claim 1, wherein the providing step comprises:
producing the biomass fraction of an aquatic species on an industrial scale; and
harvesting the biomass.

3. The process of claim 1, wherein the lysed biomass comprises a protein fraction, a carbohydrate fraction, a lipid-depleted carbohydrate fraction, a substantially lipid-free carbohydrate fraction, or a lipid fraction derived from the biomass feedstock.

4. The process of claim 1, wherein the aquatic species comprises at least one species of Lemna.

5. The process of claim 1, wherein the catalyst comprises at least one acid.

6. The process of claim 5, wherein the acid comprises at least one of HBr, HCl, HCN, HF, and H₂S.

7. The process of claim 5, wherein the acid comprises an organic acid, comprising at least one of formic acid, acetic acid, oxalic acid, and glycolic acid.

8. The process of claim 5, wherein the acid is nitric acid.

9. The process of claim 1, wherein the alcohol comprises at least one of methanol, ethanol, propanol, butanol, hexanol, heptanol, octanol, nonanol, and decanol.

10. The process of claim 1, wherein the separating step comprises pressing the lysed biomass to generate a juice.

11. The process of claim 1, wherein the separating step comprises centrifugation of the mixture.

12. The process of claim 1, wherein the recovering step comprises phase separation and fractional distillation.

13. The process of claim 10, further comprising:
mixing a material selected comprising at least one of the solid phase and the juice, with an alcohol and a catalyst to form a mixture;
further separating the mixture into a liquid fraction and a solid fraction;
whereby lipids and ash-forming components in the material are segregated into the liquid.

14. The process of claim 13, wherein the solid phase comprises at least one of:
a first solid phase generated from lysing the biomass and pressing the lysed biomass to yield a juice and first solid phase;
a second solid phase generated from filtering the juice to yield a filtered juice and a second solid phase; and
a third solid phase generated from clarifying the filtered juice to yield a clarified juice and third solid phase.

15. The process of claim 13, wherein the further separating step comprises:
subjecting the mixture to centrifugation;
collecting the solid fraction; and
recovering reagents by distillating the liquid fraction.

16. The process of claim 15, wherein the centrifugation is performed at about 3500 rpm.

17. The process of claim 15, wherein the centrifugation is performed at about 4000 rpm.

18. The process of claim 15, wherein the centrifugation is performed at about 4500 rpm.

19. The process of claim 15, wherein the centrifugation is performed for a duration of about 15 minutes.

20. The process of claim 15, wherein the centrifugation is performed for a duration of about 20 minutes.

21. The process of claim 15, wherein the centrifugation is performed for a duration of about 30 minutes.

22. The process of claim 1, further comprising a removing step to reduce ash-forming components of the biomass prior to the separating step of the process, wherein the removing step comprises:
adding the mixture to a decanting apparatus;
spinning the mixture at high speed in the decanting apparatus;
generating a solid mass retained within the decanting apparatus that is substantially freed of lipids and ash-forming components;
collecting a separated liquid that is forced through holes of the decanting apparatus and contains lipids and ash-forming components, and
further processing the separated liquid to remove ash-forming components.

23. The process of claim 10, further comprising a removing step to reduce ash-forming components from the juice generated from the pressing step of the process, wherein the removing step comprises:
adding the mixture to a decanting apparatus;
spinning the mixture at high speed in the decanting apparatus;
generating a solid mass retained within the decanting apparatus that is substantially freed of lipids and ash-forming components;
collecting a separated liquid that is forced through holes of the decanting apparatus and contains lipids and ash-forming components, and
further processing the separated liquid to remove ash-forming components.

24. The process of claim 22, wherein the mixture is generated at elevated temperature or pressure.

25. The process of claim 24, wherein the elevated temperature is about 70° C.

26. The process of claim 24, wherein the elevated temperature is about 80° C.

27. The process of claim 24, wherein the elevated temperature is about 90° C.

28. The process of claim 24, wherein the elevated pressure is about 10 psig.

29. The process of claim 24, wherein the elevated pressure is about 15 psig.

30. The process of claim 24, wherein the mixture is generated at room temperature and atmospheric pressure.

31. The process of claim 1, further comprising adding a protease enzyme to the solid fraction or solids.

32. The process of claim 1, wherein the recovering step comprises:
subjecting the liquid fraction to a filter press;
adding water to the lysed biomass;
sonicating the lysed biomass; and
adding carbohydrate enzymes to the lysed biomass individually or in combination.

33. The process of claim 32, further comprising subjecting the liquid phase or the solid phase to chromatography and solubilizing protein.

34. The process of claim 1, wherein the treating step comprises a transesterification process.

35. The process of claim 1, further comprising a leaching step, comprising:
suspending the biocrude in a solution selected from the group consisting of water and dilute acid for at least 20 hours to generate a mixture;
subjecting the mixture to a filtering system;
washing the mixture by adding water to the mixture and removing water or dilute acid; and
drying the mixture to generate a biocrude with reduced ash content.

36. A system of reducing the ash content from a biomass feedstock or of a biomass fraction of an aquatic species comprising:
a reaction chamber suitable to facilitate a reaction among the biomass feedstock, an alcohol, and a catalyst;
a vessel connected to the reaction chamber via a closable fluid connection;
a lysing unit for lysing the biomass to generate a lysed biomass;
a separating unit for separating the lysed biomass to generate a juice and a solid phase;
a separator adapted to separate the unreacted alcohol from the reaction product;
wherein processing the biomass in the system results in at least 40% reduction of the ash content of the biomass.

37. The system of claim 33, further comprising:
a first belt filter to facilitate separating a mother liquor and salts from the reaction among the biomass feedstock, the alcohol, and the catalyst;
a second belt filter to facilitate addition of wash water and removal of water and dilute acid;
a third belt filter to facilitate addition of wash water and removal of water and dilute acid;
a fourth belt filter to facilitate addition of wash water and ammonia and removal of the filtrate;
a fifth belt filter to facilitate addition of wash water and further removal of filtrate to generate a product; and
a dryer to further process the product.
